(19) United States
(12) Patent Application Publication
(10) Pub. No.: US 2015/0284794 A1
(43) Pub. Date: Oct. 8, 2015

(54) Sequencing Performance With Modified Primers

(71) Applicant: QIAGEN GmbH, Hilden (DE)

(72) Inventors: Nan Fang, Neuss (DE); Dirk Loffert, Dusseldorf (DE); Monika Ngowe, Marblehead, MA (US)

(73) Assignee: QIAGEN GmbH

(21) Appl. No.: 14/679,203

(22) Filed: Apr. 6, 2015

Related U.S. Application Data
(60) Provisional application No. 61/976,099, filed on Apr. 7, 2014.

(51) Int. Cl. C12Q 1/68

(52) U.S. Cl. CPC C12Q 1/6874

(57) ABSTRACT

Methods are described which utilize modified sequencing primers that bind to template with high specificity and stability to improve sequencing performance. In one embodiment, the method utilizes sequencing primers having 3′ and 5′ ends, comprising a minor groove binder (MGB) molecule linked to the 5′ end. In one embodiment said primer further comprises a 5′ flap and said MGB molecule is linked to the 5′ flap.

### FIELD OF INVENTION

The present invention relates generally to nucleic acid sequencing. Methods are described which utilize modified sequencing primers that bind to template with high specificity and stability to improve sequencing performance.

### BACKGROUND OF THE INVENTION

Many of the next-generation sequencing technologies use a form of sequencing by synthesis (SBS), wherein specially designed nucleotides and DNA polymerases are used to read the sequence of chip-bound, single-stranded DNA templates in a controlled manner. To attain high throughput, many millions of such template spots are arrayed across a sequencing chip and their sequence is independently read out and recorded.

There is a continued need for methods and compositions for increasing the fidelity of sequencing nucleic acid sequences.

### SUMMARY OF THE INVENTION

In a sequencing-by-synthesis reaction, specific and stable binding of the sequencing primer to template is critical for the sequencing quality. Non-specific binding of the sequencing primer to the template could cause mis-priming at the wrong start sites and consequently to the generation of background signals that interfere and compromise the sequence quality of the intended DNA sequence eventually contributing to sequencing errors. Dissociation of the sequencing primer from the template would lead to lower level of nucleotide incorporation for a given polyclonal DNA cluster and consequently lower signals that can be detected compromising read-length and contributing to a higher error rate. Furthermore, a sequencing primer with stable and specific binding on the sequencing template could be potentially more accessible to different species in the sequencing template regardless of their nucleotide composition and secondary structure and therefore reduce sequencing bias.

In one embodiment, the present invention describes modified sequencing primers that bind to template with high specificity and stability to improve sequencing performance. In one embodiment, the present invention contemplates a method for sequencing a nucleic acid by detecting the identity of a nucleotide analogue incorporated into primer extension strand in a polymerase reaction, comprising: a) providing i) sequencing primer having 3′ and 5′ ends, comprising a minor groove binder (MGB) molecule linked to the 5′ end; (ii) single-stranded template attached to a solid surface; (iii) polymerase and iv) one or more different nucleotide analogues, wherein each different nucleotide analogue comprises a base selected from the group consisting of adenine, guanine, cytosine, thymine, and uracil, and their analogues; and a unique label attached through a cleavable linker to the base or to an analogue of the base; a deoxyribose; and a cleavable chemical group at the 3′-position of the deoxyribose; 2) hybridizing the sequencing primer to the template; 3) extending the sequencing primer by incorporating a first nucleotide analogue therein with said polymerase in a polymerase reaction so as to create a primer extension strand, wherein the incorporated nucleotide analogue terminates the polymerase reaction; and 4) detecting the unique label attached to the nucleotide analogue that has been incorporated into the growing strand of DNA, so as to thereby identify the incorporated nucleotide analogue. In one embodiment, the method further comprises 5) cleaving the cleavable linker between the nucleotide analogue that was incorporated into primer extension strand and the unique label; and cleaving the cleavable chemical group at the 3′-position of the deoxyribose to leave an —OH group. In one embodiment, the method further comprises 6) extending the sequencing primer by incorporating a second nucleotide analogue therein. In one embodiment, the method further comprises 7) detecting the unique label attached to said second nucleotide analogue so as to thereby identity of the second incorporated nucleotide. In one embodiment said primer further comprises a 5′ flap and said MGB molecule is linked to the 5′ flap.

In one embodiment, the present invention contemplates a method for sequencing a nucleic acid by detecting the identity of a nucleotide analogue incorporated into primer extension strand in a polymerase reaction, comprising: a) providing i) sequencing primer having 3′ and 5′ ends, comprising a minor groove binder (MGB) molecule linked to the 5′ end, said sequencing primer hybridized to (ii) single-stranded template attached to a solid surface; (iii) polymerase and iv) one or more different nucleotide analogues, wherein each different nucleotide analogue comprises a base selected from the group consisting of adenine, guanine, cytosine, thymine, and uracil, and their analogues; and a unique label attached through a cleavable linker to the base or to an analogue of the base; a deoxyribose; and a cleavable chemical group at the 3′-position of the deoxyribose; 2) extending the sequencing primer by incorporating a first nucleotide analogue therein with said polymerase in a polymerase reaction so as to create a primer extension strand, wherein the incorporated nucleotide analogue terminates the polymerase reaction; and 3) detecting the unique label attached to the nucleotide analogue that has been incorporated into the growing strand of DNA, so as to thereby identify the incorporated nucleotide analogue. In one embodiment, the method further comprises 4) cleaving the cleavable linker between the nucleotide analogue that was incorporated into primer extension strand and the unique label; and cleaving the cleavable chemical group at the 3′-position of the deoxyribose to leave an —OH group. In one embodiment, the method further comprises 5) extending the sequencing primer by incorporating a second nucleotide analogue therein. In one embodiment, the method further comprises 6) detecting the unique label attached to said second nucleotide analogue so as to thereby identity of the second incorporated nucleotide. In one embodiment said primer further comprises a 5′ flap and said MGB molecule is linked to the 5′ flap.

In one embodiment, the present invention contemplates a primer-template complex, said template comprising single-stranded template attached to a solid surface, said primer having 3′ and 5′ ends, and comprising a minor groove binder (MGB) molecule linked to the 5′ end, and a nucleotide analogue incorporated at the 3′ end, said nucleotide analogue comprises i) a base selected from the group consisting of adenine, guanine, cytosine, thymine, and uracil, and their analogues; ii) and a unique label attached through a cleavable linker to the base or to an analogue of the base; iii) a deoxyribose; and iv) a cleavable chemical group at the 3′-position of the deoxyribose. In one embodiment of the complex, said primer further comprises a 5′ flap and said MGB molecule is linked to the 5′ flap.

### BRIEF DESCRPTION OF THE DRAWINGS

FIG. 1 shows one embodiment of a general workflow used in next generation sequencing approaches. DNA is fragmented and modified with adapters, prior to amplification in an emulsion. The emulsion is broken and the amplified (typically clonally amplified) template is sequenced.

### DEFINITIONS

Primers that contain portions non-complementary to the target are usually used to add to the PCR product a utility sequence (such as a restriction site). These non-complementary portions are often referred to as an “overhang” or as a “flap” or as a “tail.” In one embodiment, the present invention contemplates modified primers with 5′ flaps. In one embodiment, primers with short 5′ AT-rich flaps are contemplated. In one embodiment, said MGB molecule is linked to the 5′ flap of the primer.

In accordance with one embodiment of the present invention, the minor groove binder (MGB) molecule is derivatized, in essence formed into a “radical” and linked to an appropriate covalent structure or chain of atoms that attaches the minor groove binder to the primer.

### DESCRIPTION OF THE INVENTION

The present invention relates generally to nucleic acid sequencing. Methods are described which utilize modified sequencing primers that bind to template with high specificity and stability to improve sequencing performance.

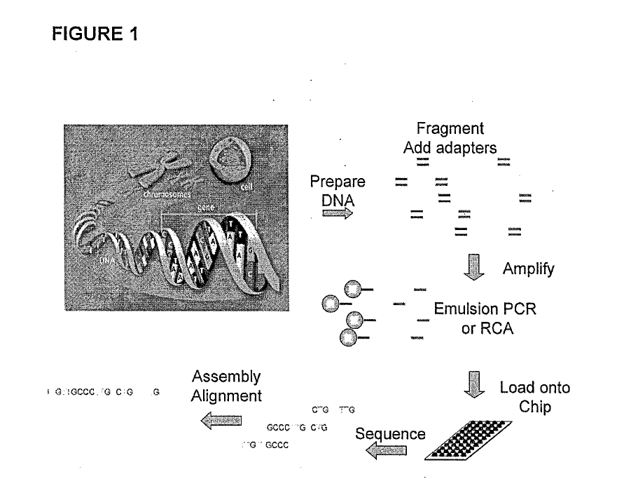

The QIAGEN GeneReader platform is a next generation sequencing (NGS) platform utilizing proprietary modified nucleotides whose 3′ OH groups are reversely terminated by a small moiety to perform sequencing-by-synthesis (SBS) in a massively parallel manner. Briefly, the sequencing templates are first clonally amplified on a solid surface (such as beads) to generate a cluster of hundreds of thousands of identical copies for each individual sequencing template. FIG. 1 shows one scheme for clonal amplification using emulsion PCR. After amplification, the double-stranded amplicon is denatured to generate single-stranded sequencing templates, hybridized with sequencing primer, and then immobilized on the flow cell. The immobilized sequencing templates are then subjected to a nucleotide incorporation reaction in a reaction mix that contains modified nucleotides with cleavable 3′ blocking group and specific fluorescent labels for each of the nucleotides A, T, C, and G. See U.S. Pat. Nos. 6,664,079, and 8,612,161 and 8,623,598, hereby all incorporated by reference. This enables the incorporation and detection of only one specific nucleotide that reversely complements the sequence on the sequencing template in each sequencing cycle and ‘reading’ of the nucleotide sequence in the sequencing template.

The sequencing primer hybridization step is critical for the sequencing performance, since specific and stable binding of the primer to the template is the prerequisite for the effective and accurate nucleotide incorporation in the sequencing cycles that follow. If the sequencing primer binds to wrong positions on the template, the wrong bases will be incorporated and called leading to increased background compromising signal to noise and eventually leading to an increased error rate. If the sequencing primer cannot bind onto the template stably and dissociates during sequencing, the signals from that specific strand will be completely lost which in turn leads to reduced signal-to-noise level and higher chance of wrong base calling.

Our method in the invention uses modified oligonucleotides as sequencing primer to facilitate specific yet stable binding to the template and therefore effective and accurate nucleotide incorporation. We tested this principle with MGB-modified sequencing primer and targeted sequencing on GeneReader platform. However, there are other types of nucleotide modifications that can enable oligos to hybridize to their target more specifically and stably, such as Locked nucleic acid (LNA), Zip nucleic acid (ZNA) [see Noir, R. et al. (2008) Oligonucleotide-oligospermine conjugates (Zip Nucleic Acids): a convenient means of finely tuning hybridization temperatures. J. Am. Chem. Soc., 130, 13500-13505], super base, or Flap sequence, among others. Such modified nucleotides are also within the scope of the invention.

The minor groove binder moiety is a radical of a molecule which can bind to the minor groove of double-stranded DNA, RNA, or DNA/RNA hybrid. See U.S. Pat. No. 6,486,308, hereby incorporated by reference. The DNA oligos with MGB modifications can form stable duplexes with their reverse complementary sequences in a second-order reaction: Due to the shorter primer sequence that is enabled by the more stable binding of the MGB-modified oligo, primer binding becomes more specificity controlled reducing the potential for mispriming. Once the primer is bound to its specific complementary sequence, the minor groove binder locks into the minor groove of the then partially double-stranded DNA molecule and stabilizes thereby the sequencing primer-template complex. Therefore, the binding of the MGB-modified oligos to the target is also more specific since any mismatch between the target sequence and MGB oligos would cause higher percentage of reduction in DNA melting temperature compared to unmodified oligos. Kutyavin I et al., Nucleic Acids Research 2000, Vol. 28, No. 2, 655-661.

### DESCRIPTION OF PREFERRED EMBODIMENTS

It is not intended that the present invention be limited to a particular MGB-modified primer. A variety of MGB compounds are known that can be attached to the 5′ end of a sequencing primer, including a 5′ flap on a sequencing primer. Examples of known minor groove binding compounds of the prior art, which can, in accordance with the present invention, be covalently bound to the 5′ end of primers to form the novel MGB-primer conjugates are certain naturally occurring compounds such as netropsin, distamycin and lexitropsin, mithramycin, chromomycin A.sub.3, olivomycin, anthramycin, sibiromycin, as well as further related antibiotics and synthetic derivatives. Certain bisquarternary ammonium heterocyclic compounds, diarylamidines such as pentamidine, stilbamidine and berenil, CC-1065 and related pyrroloindole and indole polypeptides, Hoechst 33258, 4′-6-diamidino-2-phenylindole (DAPI) as well as a number of oligopeptides consisting of naturally occurring or synthetic amino acids are minor groove binder compounds.

The minor groove binder dihydrocyclopyrroloindole tripeptide (DPI₃), folds into the minor groove formed by the terminal 5-6 bp. The crescent shaped DPI₃ is isohelical with the deep and narrow minor groove of B-form DNA where it is stabilized mainly by van der Waals forces. Increases in melting temperature (Tₘ) of as much as 49° C. were observed for A/T-rich octanucleotides.

Oligonucleotide primers with MGB molecules attached thereto are described in U.S. Pat. Nos. 7,723,038; 7,759,126; and 7,794,945, (assigned to Elitech) hereby incorporated by reference.

### EXPERIMENTAL

The GenePanel NGS library used in the tests was generated from LNCAP prostate cell line with standard QIAGEN target sequencing workflow according to manufacturer's handbooks (QIAGEN GeneRead GenePanel for Prostate Cancer (V2) and QIAGEN GeneRead Library Construction Kit.). Following library construction and qualification, 0.075 pg/μl of the GenePanel library containing GeneReader—specific adaptors was subjected to automated emulsion PCR process on GeneRead QiaCube to generated clonally amplified sequencing template on the bead surface (FIG. 1). The emulsion PCR beads were then taken out from the GeneRead QiaCube, washed twice in 1 ml PBST (PBS containing 0.05% Tween), denatured with 5000 NaOH/Tween solution (0.2N NaOH, 0.1% Tween) for 5 minutes at room temperature to generate single-stranded sequencing templates on the bead surface, divided to two parts and hybridized with either unmodified GeneReader sequencing primer, or a sequencing primer with additional 5′ flap and MGB modification.

The sequencing primer hybridization protocol is as following: the emulsion PCR beads were resuspended in 100 μl sequencing primer solution (10 μM of sequencing primer in PBST buffer), heated for 5 min 95° C. in a thermocycler, removed from the thermocycler, cooled down to RT, washed once with 500 μl PBST, twice with 500 μl phosphate buffer pH 7.5. Beads were then crosslinked onto the flow cell in 30 μl of 40 mg/ml EDC in PBST (1 hour incubation at room temperature), and subjected to sequencing on the GeneReader.

Following sequencing, the beads of each tile were analyzed with GeneReader Analyze software for mapped reads (reads mapped to the reference GenePanel sequence with less than 3 errors in the first 28 nucleotides), perfect reads (reads perfectly mapped to the reference sequence), and raw error rate at the read length of 25, 50, 75 and 100bp. The results shown in Table 1 cover 50 bp read length.

TABLE 1
MGB Modified Primer Improves Sequencing Performance.

| | | | | | | Perfect | Error |
|---|---|---|---|---|---|---|---|
| | | Mapped | Perfect | % | | Reads, | Rate, |
| Sequencing | | Reads/ | Reads/ | Per- | Error | Best | Best |
| Primer | | Tile | Tile | fect | Rate | Tile | Tile |
| Unmodified | Average | 5,258 | 336 | 5.82 | 10.87 | 1,280 | 8.67 |
| | SD | 2,200 | 245 | 1.69 | 0.72 | | |
| Modified | Average | 15,635 | 1,903 | 11.59 | 7.92 | 5,133 | 5.45 |
| with MGB | SD | 3,526 | 930 | 3.32 | 0.83 | | |

SD, Standard Deviation.
Mapped Reads, sequencing reads mapped to the reference GenePanel sequence with less than 3 errors in the first 28 nucleotides.
Perfect Reads, sequencing reads perfectly mapped to the reference sequence.
% Perfect, the percentage of perfect reads in the total mapped reads in each tile.
Error Rate: the percentage of reads not matching reference sequence in total reads.

As the sequencing results in Table 1 demonstrate, compared to the standard, unmodified sequencing primer Seq46, modified sequencing primer MGBv4 (ELITech North America, Princeton, N.J., USA) which has a MGB modification and a flap sequence on the 5′ end significantly improves sequencing performance. The average number of mapped reads in each tile is increased by about three fold and average number of perfect reads is increased by about six fold, while the error rate is reduced by about 30%.

Our results are also somewhat surprising considering the fact that oligos with MGB modification at their 5′ end was shown to arrest primer extension in PCR. Afonina I et al, Nucleic Acids Research, 1997, Vol. 25, No. 13, 2657-2660. However, in the SBS reaction on GeneReader, the modified sequencing primer with 5′ MGB and Flap clearly leads to better sequencing performance regarding read number and quality, error rate, read length, and sequencing bias.

1. A method for sequencing a nucleic acid by detecting the identity of a nucleotide analogue incorporated into primer extension strand in a polymerase reaction, comprising:
a) providing i) sequencing primer having 3′ and 5′ ends, comprising a minor groove binder (MGB) molecule linked to the 5′ end; (ii) single-stranded template attached to a solid surface; (iii) polymerase and iv) one or more different nucleotide analogues, wherein each different nucleotide analogue comprises a base selected from the group consisting of adenine, guanine, cytosine, thymine, and uracil, and their analogues; and a unique label attached through a cleavable linker to the base or to an analogue of the base; a deoxyribose; and a cleavable chemical group at the 3′-position of the deoxyribose;
2) hybridizing the sequencing primer to the template;
3) extending the sequencing primer by incorporating a first nucleotide analogue therein with said polymerase in a polymerase reaction so as to create a primer extension strand, wherein the incorporated nucleotide analogue terminates the polymerase reaction; and
4) detecting the unique label attached to the nucleotide analogue that has been incorporated into the growing strand of DNA, so as to thereby identify the incorporated nucleotide analogue.

2. The method of claim 1, further comprising 5) cleaving the cleavable linker between the nucleotide analogue that was incorporated into primer extension strand and the unique label; and cleaving the cleavable chemical group at the 3′-position of the deoxyribose to leave an —OH group.

3. The method of claim 2, further comprising 6) extending the sequencing primer by incorporating a second nucleotide analogue therein.

4. The method of claim 3, further comprising 7) detecting the unique label attached to said second nucleotide analogue so as to thereby identity of the second incorporated nucleotide.

5. The method of claim 1, wherein said primer further comprises a 5′ flap and the MGB molecule is linked to the 5′flap.

6. A primer-template complex, said template comprising single-stranded template attached to a solid surface, said primer having 3′ and 5′ ends, and comprising a minor groove binder (MGB) molecule linked to the 5′ end, and a nucleotide analogue incorporated at the 3′ end, said nucleotide analogue comprises i) a base selected from the group consisting of adenine, guanine, cytosine, thymine, and uracil, and their analogues; ii) and a unique label attached through a cleavable linker to the base or to an analogue of the base; iii) a deoxyribose; and iv) a cleavable chemical group at the 3′-position of the deoxyribose.

7. The complex of claim 6, wherein said primer further comprises a 5′ flap and the MGB molecule is linked to the 5′flap.
